(19) United States
(12) Patent Application Publication
(10) Pub. No.: US 2004/0082799 A1
(43) Pub. Date: Apr. 29, 2004

(54) Process for producing substituted epoxides

(76) Inventors: David M. Hodgson, Oxford (GB); Stephanie Lydia Madeleine Norsikian, Palaiseau (FR); Iain Donald Cameron, Manchester (GB); Emmanuel Gras, Toulouse (FR)

Correspondence Address: Nixon & Vanderhye, 8th Floor, 1100 North Glebe Road, Arlington, VA, 22201, US

(21) Appl. No.: 10/250,568

(22) PCT Filed: Dec. 14, 2001
(86) PCT No.: PCT/GB01/05559

(30) Foreign Application Priority Data
Jan. 5, 2001 (GB) 0100292.2

(51) Int. Cl. C07D 303/00

(52) U.S. Cl. 549/513

(57) ABSTRACT

A process for preparing a substituted epoxide of the formula:

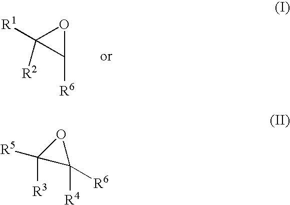

in which one of R¹ and R² is a substituent and the other is hydrogen or a substituent or R¹ and R² together complete a ring with the carbon atom to which they are attached, R³ and R⁴ which may be the same or different are substituents, or, together with the carbon atoms to which they are attached, complete a ring, R⁵ is hydrogen or a substituent and R⁶ is a substituent which comprises causing an epoxide of the formula:

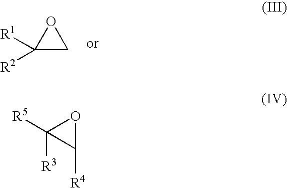

respectively, where R¹ to R⁵ are as defined above to react with an electrophile with the aid of an organolithium compound and a ligand which is an at least bicyclic compound comprising 2 ring nitrogen atoms, said nitrogen atoms being tertiary.

This invention relates to a process for producing substituted epoxides from epoxides which do not possess an activating substituent.

Epoxides are widely utilized as versatile synthetic intermediates. Their reactions are dominated by the electrophilic nature of the epoxide, generally involve cleavage of the strained three-membered ring and include a wide range of nucleophilic ring openings and acid-base-induced isomerization reactions. In contrast, the utility of epoxides as nucleophiles (via oxiranyl anions) is less developed, although such reactions can provide a very direct way to assemble substituted epoxides. A significant current limitation with this strategy is the apparent necessity of using epoxides possessing electron-withdrawing or trialkylsilyl or trialkylstannyl groups attached to the epoxide ring. Such epoxide substrates may not be readily available and the activating group may not be wanted in the product; both of these issues detract from the utility of the method. Electron-withdrawing and trialkylsilyl substituents facilitate formation of oxiranyl anions by promoting deprotonation (usually lithiation) and prolonging the solution lifetime of these otherwise very labile intermediates. Trialkylstannyl- and sulfinyl-substituted epoxides react with organolithium (by transmetallation and desulfinylation respectively) rapidly enough at low temperatures such that the resultant unstabilized oxiranyl anions can exhibit synthetically useful nucleophilic (rather than carbene-type) reactivity.

The present invention is based on the finding that the presence of appropriate ligands can serve the dual role of accelerating deprotonation and reducing the rate of oxiranyl anion decomposition such that electrophile trapping of unfunctionalized epoxides is possible.

Accordingly the present invention provides a process for preparing a substituted epoxide of the formula:

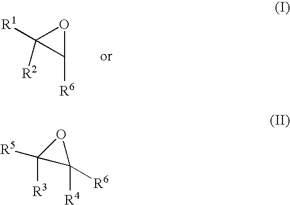

in which one of R¹ and R² is a substituent and the other is hydrogen or a substituent, or R¹ and R² together complete a ring with the carbon atom to which they are attached, R³ and R⁴, which may be the same or different, are substituents, or, together with the carbon atoms to which they are attached, complete a ring, R⁵ is hydrogen or a substituent and R⁶ is a substituent, which process comprises causing an epoxide of the formula:

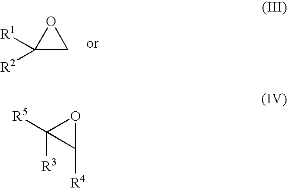

respectively, where R¹ to R³ are as defined above to react with an electrophile with the aid of an organolithium compound and a ligand which is an at least bicyclic compound possessing 2 ring nitrogen atoms, said nitrogen atoms being tertiary.

Thus, the process of the present invention starts with either a terminal epoxide of formula (III) or a 1,2-disubstituted epoxide of formula (IV) (although there can be a third substituent). It will be appreciated that the terminal epoxides are generally readily available since they can be obtained from the conversion of the corresponding olefins.

The nature of the substituents present is generally unimportant, although it is preferred to exclude any substituent which is sensitive to base or is susceptible to nucleophilic attack since this can result in the organolithium reacting with this substituent rather than at the desired epoxide position. Thus, in general, the presence of carbonyl group-containing substituents derived from aldehydes, ketones and esters should be avoided, although it is possible to protect them with base-stable protecting groups.

Thus, suitable substituents include aliphatic groups which may be unsaturated and can be substituted and aromatic groups such as an aryl group, as well as halogen, alkyl or aryl ether or silyl ether.

As used herein, an alkyl group is typically a linear or branched alkyl group containing from 1 to 6 carbon atoms, such as a C₁-C₄ alkyl group, for example methyl, ethyl, n-propyl, i-propyl, n-butyl, i-butyl and t-butyl or a higher alkyl group having 6 to 18, typically 6, 8 or 10, carbon atoms.

An alkyl group may be unsubstituted or substituted at any position. Typically, it is unsubstituted or carries one or two substituents. Suitable substituents include aryl, halogen and silyl-oxy, such as t-BuMe₂SiO.

Unsaturated aliphatic groups include ethylenically unsaturated groups having, typically, the same number of atoms and substituents as indicated above for the alkyl group, for example CH₂═CH(CH₂)₆—; the unsaturation may be at the end of the alkyl chain i.e. as a vinyl substituent.

As used herein, an aryl group is typically a C₆-C¹⁰ aryl group such as phenyl or naphthyl. Phenyl is preferred. An aryl group may be unsubstituted or substituted at any position. Typically, it carries 1, 2, 3 or 4 substituents. Suitable substituents include aryl, heteroaryl and heterocyclic groups, halogen, alkyl, for example haloalkyl, alkylthio, alkoxy, for example haloalkoxy and hydroxy.

An aryl group may optionally be fused to a further said aryl group or to a carbocyclic, heterocyclic or heteroaryl group. For example, it may be fused to a pyridine ring to form a quinoline group, or to a 1,4 dioxane or 1,3 dioxolane ring.

As used herein, a silyl group is typically a tri(alkyl/aryl) silyl group i.e. a silyl group substituted bay 3 alkyl and/or aryl groups, typically by 3 alkyl groups, which is preferred, or 3 aryl groups. The alkyl (and aryl) groups need not all be the same. A specific example is t-butyl, dimethylsilyl.

As used herein, a halogen is typically chlorine, fluorine or bromine

As used herein, a heterocyclic group is typically a non-aromatic, saturated or unsaturated C5-C₁₀ carbocyclic ring in which one or more, for example 1, 2 or 3, of the carbon atoms are replaced by a heteroatom selected from N, O and S. Saturated heterocyclic groups are preferred. Examples of suitable heterocyclic groups include piperidine morpholine, 1,4-dioxane and 1,3-dioxolane.

A heterocyclic group may be unsubstituted or substituted at any position. Suitable substituents include nitro, halogen, alkyl, for example haloalkyl, alkylthio, alkoxy, for example haloalkoxy or hydroxy.

As indicated above, R¹ and R² can together form, with the carbon atom to which they are attached, a cyclic group, typically a carbocyclic group such as a cycloaliphatic group, typically of 3 to 18 carbon atoms, more particularly 5 to 14 carbon atoms, more particularly 12 carbon atoms as in cyclododecyl. Likewise R³ and R⁴ can together form, with the carbon atoms to which they are attached, a ring, generally 7- or 8-membered, typically a carbocyclic ring.

In one embodiment, the substituents are not activating groups (i.e. groups which promote substitution on the carbon atom to which they are attached) as used in the past. These are essentially of two types, namely those which are eliminated during the reaction and replaced, and those which remain but activate substitution of the carbon atom to which they are attached. Substituents of the first type include sulfones, hydrocarbonoxysulfonyl and hydrocarbyl tin. The second group are generally anion stabilising groups such as trihydrocarbylsilicon ester, cyano and phenyl groups. Of course, the presence of this latter type should not necessarily be excluded if the atom to which the substituent is attached is already substituted.

The process of the present invention involves lithiation using an organolithium compound. Suitable such compounds include alkyllithium compounds, especially where the alkyl groups are of one to six, preferably at least three carbon atoms, for example three or four carbon atoms, and preferably branched alkyl groups such as iso-propyl and secondary butyl.

A key to the success of the reaction involves the presence of a ligand which is preferably the specified cyclic diamine. The compound must usually be such as will provide two coordinating atoms for binding to the lithium. As indicated, the amino groups, if they do not form part of a bridge or an unsaturated linkage are substituted so that they are tertiary. Suitable substituents include an aliphatic or aromatic group, preferably an alkyl group, typically of one to six carbon atoms, especially one to four carbon atoms, such as methyl or n-butyl. Typical such amines which can be used in the process of the present invention are to be found in, for example, Hoppe et al, Angew. Chem. International Edition in English 1997, 36, 2282-2316; Zefirov, Topics Stereochemist, 20, 1991 Mukaiyama et al, Topics in Current Chemistry 1985 127:133-167, and Togrui et al, Angew. Chem.—International Edition in English 194 33: 497-526,. More particularly, the cyclic compound may be a bridged or unbridged compound. Typical unbridged compounds include those having the following skeletons:

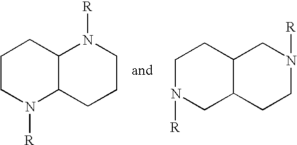

where each R group, which may be the same or different, represents the specified substituent. It is generally preferred that the rings are cis-fused. Of course, the presence of other fused rings as well as substituents is not excluded.

Examples of bridged structures which can be used in the present invention include those with the following skeletons:

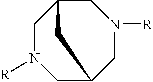

where R is as defined above. Further fused rings can be present. In particular, the R groups can complete a ring with either of the carbon atoms adjacent to the nitrogen atom to which it is attached. Typically, a five or six membered saturated ring which is generally carbocyclic is formed in this way.

The use of these bridged amines is particularly preferred. Specific examples are illustrated below.

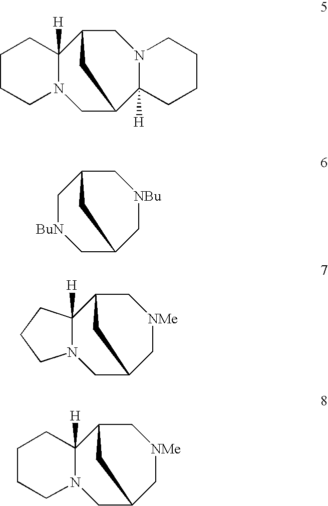

A wide variety of electrophiles can be reacted with the epoxides in accordance with the process of the present invention to introduce the substituent R₆. Many of the electrophiles will be halides whereby the moiety attached to the halide atom forms the substituent (R₆) in the epoxide. Suitable halides which can be used include alkyl halides, typically of one to twelve, for example one to six carbon atoms such as methyl, ethyl and butyl. The aliphatic groups are preferably straight chain although they can also be branched. Also, the aliphatic groups can be unsaturated, in general ethylenically unsaturated, as in, for example, alkenyl. Other electrophilic halides which can be used in the process include silicon and tin halides, in general substituted by three hydrocarbon groups generally alkyl or aryl groups, for example those listed in connection with the alkyl halides, such as trialkyl, e.g. trimethyl, silicon and tin. A specific example of such an electrophile is trimethyl tin chloride (Me₃SnCl). Other halides which can be used include sulfonyl and phosphoryl halides. The halides are typically chlorides or iodides e.g. methyl iodide. Alternatively other groupings such as trifluoromethane sulfonate and can be employed in place of the halides.

Apart from these electrophiles it is also possible to use a variety of carbonyl compounds, including aldehydes and ketones which give rise to the formation of a hydroxy substituent, as well as esters, amides and chloroformates which give rise to ketone or ester substituents. The carbonyl compound can be aliphatic and/or aromatic and can contain the substituents listed above. A particular example is benzaldehyde which gives rise to a Ph—CH(OH)-substituent.

A further advantage of the process of the present invention is that it is stereoselective and generally highly stereoselective. As might have been expected, with a terminal epoxide, substitution takes place on the unsubstituted carbon atom in the position trans- to the original substituent. Surprisingly, it has been found that this substitution is very clean and gives this particular isomer almost, if not entirely, exclusively.

It will also be appreciated that for all terminal epoxides there is more than one chiral form, so that the starting material can either be racemic or in the form of an individual enantiomer. Generally, the best results have been obtained using the ligand (−)-sparteine, which is readily obtainable. It has been found that using a particular enantiomeric form of the ligand results in a preferential reaction with one enantiomeric form of the epoxide. In other words, even if one uses a racemic epoxide, by using a particular enantiomeric form of the ligand it is possible to obtain kinetic resolution with the result that the product is enriched in one particular enantiomer.

Typically, the 1,2-disubstituted epoxides readily available are symmetrical i.e. the two substituents are the same, and preferably cis—the epoxide is then achiral. Again if one uses a particular enantiomeric form of the ligand then an enantioselective synthesis results i.e. the resulting substituted epoxide is obtained with a predominance of one of the two enantiomeric forms.

It will be appreciated that when the starting material is of formula (IV) and R⁵ is hydrogen, in general, substitution can occur at either carbon atom. Thus it is to be understood that in formula (II) the substituted group can be either at R⁵ or at R⁶

The process of the present invention is typically carried out in a solvent. Suitable solvents include ethereal solvents such as dialkyl ethers, for example diethyl ether, as well as cyclic ethers such as tetrahydrofuran. In addition, hydrocarbon solvents can also be used. These can be aliphatic such as hexane, which is particularly preferred, and pentane, or aromatic such as cumene.

Due to the instability of the intermediate it is necessary to carry out the lithiation reaction at low temperature, as is well known in the art. In general, temperatures from −78° C. to −90° C. are suitable. Thus, typically, a solution of the ligand in the solvent is mixed with the organolithium compound in the solvent and then cooled and the epoxide added along with, or followed by, the electrophile, typically as a solution. Thus, in one embodiment, the epoxide reacts with the electrophile in the presence of the organolithium compound and ligand. As one of skill in the art will know, lithiation reactions can be very quick or they can take up to, say, two hours. The substitution with the electrophile can also take an hour or more so that, in general, the reaction time is from one to five hours, for example 2 to 4.5 hours. After the reaction, the desired product can be worked up in a standard way, typically using methanol and, generally, dilute aqueous hydrochloric acid, or phosphoric acid or a saturated ammonium chloride solution.

Typically, roughly equimolar amounts of organolithium and ligand are employed and also roughly equimolar amounts of the epoxide and the electrophile. In general, 1 to 2.5 times the stoichiometric amount of organolithium compound and likewise of the ligand can generally be employed, although if the electrophile is one that activates, as with trimethylsilyl chloride the amount of organolithium compound should be kept to the stoichiometric amount to avoid disubstitution. In some circumstances the use of an equimolar amount, or slight excess of the base, for example up to 1.24 or 1.5 moles per mole of epoxide is desirable since a significant excess can destabilize the intermediate. On the other hand, the use of at least double the quantity of organolithium compound does have a particular utility in the case of terminal epoxides. As indicated above, the process of the present invention gives rise to the trans-isomer. However, it is also possible to obtain the cis-isomer. If the electrophile is chosen such that the substituent formed is an activating substituent, ten this substituent will be introduced in the transposition and a second equivalent of organolithium will subsequently react (by deprotonation) at the vacant cis-position. If one then adds the electrophile corresponding to the desired substituent to this product, the desired substituent will be introduced in the cis-position It is then a simple matter to eliminate the trialkylsilyl substituent, for example by the addition of fluoride ions such as in an alkali metal fluoride, typically potassium fluoride or, for example, tetrabutyl ammonium fluoride and then one is left with a product with the substituent in the cis-position.

It will be appreciated that this contrasts with the prior art procedure where it is first necessary to introduce the activating group by some alternative means, to isolate the product and then to subject it to a second reaction to introduce the desired substituent. In accordance with the present invention, the whole procedure can be carried out without the need to isolate the compound with the activating substituent.

The following Examples further illustrate the present invention.

### EXAMPLE 1

In order to assess the feasibility of electrophilic trapping, deuteration of a simple terminal epoxide was tried. The first indications that D incorporation is possible were found using (−)-sparteine (5) as the ligand. 15 Min after addition of 1 (1,2-epoxydodecane) to a mixture of 5 and n-BuLi, t-BuLi or i-PrLi in diethyl ether at −90° C., resulted in 1 with 0% D, 10% D and 22% D incorporation, respectively. After 1 h at −90° C. with i-PrLi/5 in diethyl ether, 1 was recovered (50% yield) with 45% D incorporation, along with 3 (18%) and 4 (R=i-Pr, 11%).

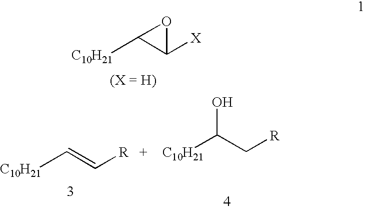

D Was incorporated exclusively trans to the alkyl substituent on the oxirane ring. Switching to hexane as solvent gave, after 1 h at −90° C. with i-PrLi/5 (Table 1, entry 1), a similar level of D incorporation in 1 to that observed using diethyl ether, but significantly less 3 as formed (6%). no 4 was observed, and more epoxide was recovered (60%)

TABLE 1
Effect of experimental conditions on the lithiation-deuteration of 1,2-
epoxydodecane (1) in hexane at −90° C.

| | | | | | Yield of 1 |
|---|---|---|---|---|---|
| Entry | RLi | Ligand | Time (min) | % D^{b} in 1 | (%)^{c} |
| 1 | i-PrLi | 5 | 60 | 46 | 60 |
| 2 | i-PrLi | 5 | 180 | 50 | 50 |
| 3 | s-BuLi | 5 | 15 | 75 | 70 |
| 4^{d} | s-BuLi | 5 | 60 | 90 | 40 |
| 5 | s-BuLi | 6 | 15 | 45 | 82 |
| 6 | s-BuLi | 7 | 15 | 50 | 85 |
| 7 | s-BuLi | 7 | 60 | 52 | 80 |
| 8 | s-BuLi | 8 | 15 | 63 | 91 |
| 9 | s-BuLi | 8 | 60 | 70 | 75 |

Reactons were carried out by addition over 10 min of a solutin of 1 (1 equiv) in hexane to a mixture of RLi/ligand (2.5 equiv each) in hexane at −90° C. followed by addition of MeOD after the time indicated. ^{b}Determined by ¹H NMR. ^{c}Isolated yields after chromatography. ^{d}4 (R = s-Bu) was only observed (5%) in entry 4.

The most encouraging results with 5 were obtained with s-BuLi in hexane at −90° C., which after 15 min gave 1 in 70% yield with 75% D incorporation (Table 1, entry 3). In this case, only 9% of 3 was isolated. Longer reaction times resulted in diminished recovery of 1 (Table 1, entries 2 and 4). The success with 5 led to examination of 6-8, which all possess the 3,7-diazabicyclo[3.3.1]nonane structural feature of 5 (Table 1, entries 5-8). For 6-8 the deprotonation step was slower than with 5. The best results were obtained with s-BuLi/8 in hexane at −90° C. (Table 1, entries 8 and 9); using s-BuLi/8 in diethyl ether was less effective giving after 1 h a mixture of 1 and 3 (1:3, 96:4), with only 48% D incorporation in 1.

### EXAMPLE 2

Trimethylsilyl substitution was achieved when trimethylsilyl chloride (TMSCl) is present during the generation of the oxiranyl anion (Scheme 2, Table 2). α,β-Epoxysilanes 10 are especially valuable in organic synthesis since, for example, they can be hydrolysed to give carbonyl compounds, undergo regioselective and stereospecific ring-opening with a range of nucleophiles to give substituted β-hydroxysilanes and are used as vinyl cation equivalents.

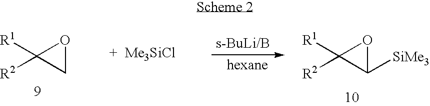

The results are shown in Table 2. These indicate that the process is compatible with a range of functionalised epoxides leading to trans-α,β-epoxysilanes (entries 2-5). The reaction is also applicable to the preparation of trisubstituted epoxies (entries 6 and 7). For the unsymmetrical epoxide in entry 7, silylation occurred with a high degree of regioselectivity (97/3) trans to the phenyl substituent.

TABLE 2
Direct synthesis of α,β-epoxysilanes from epoxides.

| | | Time | 10 |
|---|---|---|---|
| Entry^{a} | 9 | (h) | (Yield, %)^{b} |
| 1 | 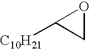 | 2 | 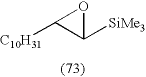 |
| 2 | 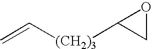 | 2.5 | 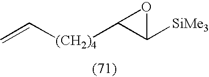 |
| 3 | 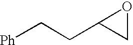 | 4.5 | 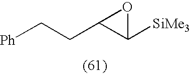 |
| 4 | 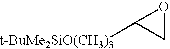 | 2 | 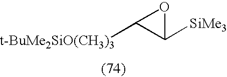 |
| 5 | 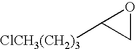 | 3 | 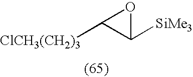 |
| 6 | 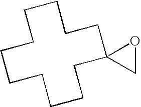 | 4.5 | 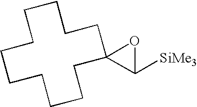 |
| 7 | 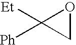 | 3 | 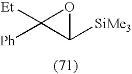 |

^{a}Reactions were carried out at −90° C. (−83° C. for entry 3) for the time indicated, followed by warming to −80° C. over 5 min (to −50° C. over 30 min for entries 1 and 7).
^{b}Isolated yield of 10 after chromatography.

The epoxides used were commercially available or prepared according to: (a) Ellings, J. A.; Downing, R. S.; Sheldon, R. A. Eur. J. Org. Chem. 1999, 837-846 (entry 3). (b) Yang, L.; Weber, A. E.; Greenlee, W. J; Patchett, A. A. Tetrahedron Lett. 1993, 34 7035-7038 (entry 4). (c) Rothberg, I; Schneider, L; Kirsch, S; OFee, R. J. Org. Chem. 1982, 47, 2675-2676 (entry 5). (d) Michnick, T. J.; Matteson, D. S. Synlert 1991, 631-632 (entries 6-7).

A typical procedure for the α,β-epoxysilane preparation was as follows:

A solution of 8 (116 mg, 0.60 mmol) in hexane (1 mL) was added to a stirred solution of s-BuLi (1.3 M in cyclohexane, 0.45 mL, 0.59 mmol) in hexane (4 mL) at −90° C. and the reaction mixture was then allowed to warm to 0° C. over 15 min. After a few seconds at 0° C., the mixture was recooled to −90° C. and a solution of 1,2-epoxydodecane (44.1 mg, 0.24 mmol) and TMSCI (36 μl, 0.28 mmol) in hexane (1 mL) was added dropwise over 10 min. After the reaction mixture had been stirred for 2 h at −90° C., it was allowed to warm slowly to −50° C. over 30 min and then MeOH (1 mL) was added, followed by 1N HCl (2 mL) at 0° C. The two phases were separated and the aqueous layer was extracted with Et₂O (2×5 mL). The combined organic extracts were washed with brine (1×5 mL), dried (MgSO₄) and concentrated under reduced pressure. The residue was purified by column chromatography on silica gel (pentane/diethyl ether: 99.5/0.5) to give 45.1 mg of 10 (R²=C₁₀H₂₁, R¹=H, 73% yield): R_{ƒ}=0.3 (pentane); [α]D²⁵=−3.8 (1.1, CHCl₃), IR (neat) 2957, 2925, 2854, 1467, 1249, 848 cm⁻¹; ¹H NMR (400 MHz, CDCl₃) δ2.76-2.74 (m. 1H), 1.97 (d, 1H,J=3.5 Hz), 1.62-1.26 (m, 18H), 0.88 (t, 3H, J=7 Hz), 0.06 (s, 9H); ¹³C NMR (100 MHz, CDCl₃) δ56.2, 51.7, 34.1, 31.9, 29.6, 29.5, 29.3, 26.4, 22.7, 14.1, −3.7; CIMS m/z (relative intensity) 257 (M+H³⁰ , 15), 129 (15), 90 (100), 73 (15); HRMS calcd for C₁₅H₃₂OSi 257.2300 found 257.2300.

The same procedure with (S) 1,2-epoxydodecane afforded trans (1R), 2S)1-(trimethylsilyl)-1,2-epoxydodecane with 72% yield. [α]D²⁵=−12.5 (1,1, CHCl₃).

Other compounds were prepared in a similar manner with the following characterising data:

trans 4phenyl-1-(trimethylsilyl)-1-epoxybutene oxide (Table 2, entry 3)

A colorless oil. ¹H NMR (400 MHz, CDCl₃, 25° C.): δ=7.35-7.1 (m, 5H; Ph), 2.95-2.75 (m, 3H; CH and CH₂), 2.1-1.8 (m, 3H; CH and CH₂), 0.04 (s, 9H; SiMe₃); ¹³C NMR (50 MHz CDCl₃, 25° C.): δ=141.9 (q), 128.9 (CH), 128.8 (CH), 128.7 (CH), 126.4 (CH), 56.1 (CH), 52.5 (CH), 36.3 (CH₂), 33.1 (CH_{2),} 3.2 (SiMe₃); IR (neat): ν=3017, 2975, 2954, 2943, 2858, 1604, 1496, 1454, 1416, 1292, 1249, 1031, 864, 840, 747, 699 cm⁻¹; MS (CI): m/z (%) 238 (50) [M+NH₄⁺], 90 (100) (Found: M+NH₄⁺, 238.1625. C₁₃H₂₀OSi, M requires M+NH₄⁺, 238.1627).

trans t-butyldimethylsilyl ether of 1-(trimethylsilyl)-1-epoxypenten-5-ol oxide (Table 2, entry 4):

A colorless oil. ¹H NMR (400 MHz, CDCl₃, 25° C.): δ=3.75-3.6 (m, 2H; CH₂), 2.85-75 (m, 1H; CH), 1.98 (d, ³J(H,H)=3.5 Hz, 1H; CH), 1.8-1.6 (m, 4H; 2×CH₂), 0.89 (s, 9H; CH₃) 0.07 (s, 15H; SiMe₃): ¹³C NMR (100 MHz, CDCl₃, 25° C.): δ=68.3 (CH₂, 55.6 (CH), 51.2 (CH), 30.0 (CH₂), 29.9 (CH₂), 25.6 (CH₂), 18.3 (q), −3.4 (SiMe₃), −5.53 (SiMe₃); IR (neat): ν=2970, 2960, 2930, 2856, 1470, 1247, 1097, 838, 775 cm⁻¹; MS (CI); m/z (%) 289 (95) M+H⁺], 273 (100), 217 (10), 164(10), 132 (20), 90 (90) (Found: M+H⁺, 289.2023. C₁₄H₃₂O₂Si₂, M requires M+H⁺, 289.2019).

trans 6-chloro -1-(trimethylsilyl)-1-epoxyhexene oxide (Table 2, entry 5)

A colorless oil. ¹H NMR (400 MHz, CDCl₃, 25° C.): δ=3.56 (t, ³J(H,H)=6.3 Hz, 2H; CH₂), 2.8-2.75 (m, 1H; CH), 1.98 (d, ³J(H,H)=3.6 Hz, 1H; CH), 1.9-1.85 (m, 2H; CH₂), 1.8-1.5 (m, 4H; 2×CH₂), 0.07 (s 9H: SiMe₃); ¹³C NMR (100 MHz, CDCl₃, 25° C.): δ=55.8 (CH), 51.5 (CH), 44.8 (CH₂), 33.2 (CH₂), 32.2 (CH₂), 23.7 (CH₂), −3.9 (SiMe₃); IR (neat): ν=2969, 2858, 1456, 1418, 1289, 876, 839 cm⁻¹; MS (CI): m/z (%) 224 (50) [M+NH₄⁺], 90(100) (Found: M+NH₄⁺, 224.1232. C₉H₁₉OSiCl, M requires M+NH+, 224.11237).

### EXAMPLE 3

Double Functionalisation

To a solution of ^{s}BuLi (1.3 M in cyclohexane, 1 mL 1.3 mmol) in hexane (8 mL) was added at −90° C. (−) sparteine (0.3 mL, 1.3 mmol) and the temperature of the mixture was allowed to warm to 0° C. for 5 min. After cooling again at −90° C., a solution of 1,2-epoxydodecane (184 mg, 1 mmol) and TMSCl (0.16 mL, 1.3 mmol) in ether (2 mL) was added dropwise over a period of 15 min and the mixture was stirred at −90° C. for 15 min. The reaction was allowed to warm slowly over 15 min to −50° C. and then recooled to −90° C. and THF (8 mL) and further TMSCl (0.25 mL, 2 mmol) and ^{s}Bu Li (1.3 M in cyclohexane, 1.5 mL, 2 mmol) added. After 15 min at −90° C. the rection was allowed to warm to room temperature overnight and then quenched with 1N HCl (5 mL). The layers were separated, the aqueous layer was extracted twice with ether (5 mL), the combined organic phases were washed with brine (5 mL), dried MgSO₄ and solvents were removed under reduced pressure. Purification by column chromatography (SiO₂, pentane) gave (I)R¹=SiMe₃, R²=SiMe₃, R⁶=C₁₀H₂₁, (170 mg, 52%); R_{f} 0.3 (pentane); vmax(neat)/cm⁻¹ 2956, 2925, 2854, 1467, 1250, 1051, 841, 812, 762, 721, 687; δH(200 MHz) 2.96 (1H, m, epoxide CH₂), 1.7-1.1 (18 H, m, 9×CH₂) 0.89 (3H, t, J 6.8, CH₃), 0.13 (9H, s, SiMe₃) and 0.06 (9H, s, SiMe₃); δC (50 MHz) 62.4 (CH), 51.4 (quat), 32.4 (CH₂), 32.0 (CH₂), 30.0 (CH₂), 29.8 (CH₂), 28.0 (CH₂), 26.8 (CH₂), 23.1 (CH₂), 14.6 (CH₃), 0.7 (SiMe₃) and −1.68 (SiMe₃); m/z (Cl) 329 (M+H⁺, 35%), 255 (20), 147 (20), 147 (20) 90 (100), 73 (70) (Found: M+H⁺, 329.2698. C₁₅H₃₁OSi₂, M requires 329.2696.

### EXAMPLE 4

Stannylation of Cyclooctene Oxide

To a solution of sBuLi (1.4 M in cyclohexane, 1.8 mL, 2.5 mmol) in Et₂O (8 mL) was added dropwise at −90° C. (−)-sparteine (596 μL, 2.6 mmol). After one hour at this temperature a precooled solution of cyclooctene oxide (252 mg, 2.0 mmol) in Et₂O (2 mL) was added rapidly and the mixture was stirred at −90° C. for two hours. Bu₃SnCl (705 μL, 2.6 mmol) was then added dropwise at −90 C. The reaction was allowed to warm to room temperature. After quenching with 0.5 M H₃PO₄ (25 mL), the organic phase was washed with saturated aqueous NaHCO₃ (25 mL) and brine (25 mL). The aqueous layers were extracted twice with Et₂O (25 mL) and the combined organic phases were dried (MgSO₄) and evaporated under reduced pressure. Purification of the residue by column chromatography on silica gel (Pet. Spirit/ether:99/1) gave I (R⁵=SnBu₃, R³,R⁴=C₆H₁₂, completing a ring) 502 mg (60%) as a colorless oil.

R_{f} (Pet Spirit/Et₂O:9/1) 0.8; IR (neat, cm⁻¹) 2958 (s); 2919 (s); 2848 (m); 1472 (m); 1456 (m); 1413 (m); 1373 (m); 1070 (m); 1019 (m); 921 (m); 736 (m); 685 (m); 661 (m); M.S. (FAB+, m/z, relative intensities): 416(25%); 359 (85); 291 (95); 235 (50); 179 (100); 135 (35);

α_{D}²⁴ (c=1.0, CHCl₃) −38.2; HRMS for M⁺ Calculated: 416.2101 Measured :416.2098; NMR ¹H (CDCl₃, 500 MHz): 2.81 (dd, J 10, 4.5, 1H); 2.18-2.15 (m, 2H); 1.63-1.33 (m, 23H); 0.96-0.91 (m, 14H); NMR ¹³C (CDCl₃, 50 MHz): 62.5 (Cq); 59.4 (epoxide CH); 32.3 (CH₂); 29.1 (CH₂); 27.4 (CH₂); 26.9 (CH₂); 26.7 (CH₂); 26.6 (CH₂); 26.2 (CH₂); 25.8 (CH₂); 13.6 (CH₃); 8.8 (SnCH₂) NMR ¹¹⁹Sn (CDCl₃, 186 MHz) −26.0

### Example 5

Ethylcarbonylation of cyclooctene oxide

To a solution of sBuLi (1.4 M in cyclohexane 1.8 mL, 2.5 mmol) in Et₂O (8 mL) was added dropwise at −90° C. (−)-sparteine (596 μL, 2.6 mmol). After one hour at this temperature, a precooled solution of cyclooctene oxide (252 mg, 2.0 mmol) in Et₂O (2 mL) was added rapidly and the mixture was stirred at −90° C. for two hours. N,N-dimethylpropionamide (330 μL, 3.0 mmol) was then added dropwise at −90° C. The reaction was allowed to warm to room temperature. After quenching with 0.5 M H₃PO₄ (25 mL), the organic phase was washed with saturated aqueous NaHCO₃ (25 mL) and brine (25 mL). The aqueous layers were extracted twice with Et₂O (25 mL) and the combined organic phases were dried (MgSO₄) and evaporated under reduced pressure. Purification of the residue by column chromatography on silica gel (Pet. Spirit/ether: 99/1) gave I (R¹═C₂H₅C═O, R²R⁶═C₆H₁₂, completing a ring). 249 mg (68%) as a colorless oil.

R_{f} (Pet Spirit/ether: 9/1) 0.6; α_{D}²⁴ (c=1.0, CHCl₃) +27.5; IR (neat, cm⁻¹) 2974 (s);

2919 (s);2856 (m); 1708 (s); 1464 (m); 1448 (m); 1404 (m); 1117 (m); 1027 (m); 924 (m); M.S. (C.I. m/z, relative intensities): 200 (12%); 183 (7); 167 (100); 137 (20); HRMS for MNH₄⁺ Calculated: 200.1651; Measured: 200.1653; NMR ¹H (CDCl₃, 200 MHz): 3.03 (dd, J 9.5, 3.8, 1H, epoxide CH); 2.51-2.20 (m, 4H, COCH₂+2H α to the epoxide); 1.61-1.24 (m, 10H, 5CH.); 0.94 (t, J 7.3, 3H, CH₃); NMR ¹³C (CDCl₃, 50 MHz): 210.8 (CO); 64.1 (Cq); 60.4 (epoxide CH); 30.2 (CH₂; 28.9 (CH₂); 27.87 (CH₂; 26.6 (CH₂); 26.4 (CH₂); 26.1 (CH₂); 24.6 (CH₂); 7.4 (CH₃); e.e. 79% (chiral GC, Chrompack Chirasil DEX-CD, 130° C., 0.7 ml/min) t_{R}mn: 8.19 t_{R}maj 9.38

1. Process for preparing a substituted epoxide of the formula: 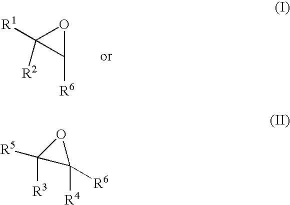
in which one of R¹ and R² is a substituent and the other is hydrogen or a substituent or R¹ and R² together complete a ring with the carbon atom to which they are attached R³ and R⁴ which may be the same or different, are substituents, or, together with the carbon atoms to which they are attached, complete a ring, R⁵ is hydrogen or a substituent and R⁶ is a substituent which comprises causing an epoxide of the formula: 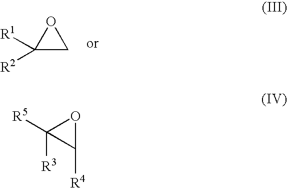
respectively, where R¹ to R⁵ are as defined above, to react with an electrophile with the aid of an organolithium compound and a ligand which is an at least bicyclic compound possessing 2 ring nitrogen atoms, said nitrogen atoms being tertiary

2. Process according to claim 1 in which the ligand is an at least bicyclic compound comprising 2 ring nitrogen atoms, said nitrogen atoms being tertiary.

3. Process according to claim 2 in which the ligand has the formula: 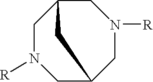
where R represents an alkyl group of 1 to 6 carbon atoms or R completes with the nitrogen atom to which it is attached and a carbon atom adjacent to the nitrogen atom a 5 or 6 membered saturated carbocyclic ring.

4. Process according to any one of claims 1 to 3 in which the ligand is a single enantiomeric form.

5. Process according to claim 4 in which the ligand is (−)-sparteine.

6. Process according to any one of the preceding claims in which the organolithium compound is a branched alkyllithium compound.

7. Process according to claim 6 in which the organolithium compound is isopropyl or secondary butyllithium.

8. Process according to any one of the preceding claims in which the electrophile is an alkyl or aryl halide or an alkylsilyl halide.

9. Process according to claim 8 in which the electrophile is trimethylsilyl chloride.

10. Process according to any one of the preceding claims in which at least one of R¹, R², R³, R⁴ and R⁵ is an alkyl group, optionally substituted by an aryl, trialkylsilyl, halide or vinyl group or is a cycloalkyl group.

11. Process according to any one of the preceding claims in which the epoxide is of formula (III).

12. Process according to claim 11 in which the epoxide of formula (III) is racemic, the ligand is a specific enantiomer such that the substituted epoxide (I) is enriched in one enantiomer.

13. Process according to claim 11 in which the electrophile provides an anion stabilising group, at least two moles of organolithium compound are used per mole of epoxide and the substituted epoxide is of formula (II) where R⁴ and R⁶ represent the same anion stabilising group.

14. Process according to claim 11 which comprises reacting the epoxide of formula (III) with a molar amount of an electrophile providing an anion stabilising group and at least 2 moles of organolithium compound and the product formed is reacted with a further electrophile and the anion stabilising group is removed to leave a compound of formula (I) with the substituent R⁶ is the cis position.

15. Process according to claim 14 in which the anion stabilising group is a trihydrocarbyl silicon group which is removed by the addition of fluoride ions.

16. Process according to any one of claims 1 to 10 in which the epoxide material is of formula (IV) in which R⁵ is hydrogen and R³ and R⁴ are the same and, the ligand is a specific enantiomer such that the substituted epoxide of formula (II) is enriched in one particular enantiomeric form.

17. Process according to claim 1 substantially as described in any one of the Examples.

18. A substituted epoxide of formula (I) or (II) as defined in claim 1 whenever prepared by a process as claimed in any one of claims 1 to 17.
